SEQUENCE LISTING (7 sequences)

SEQ ID NO: 1 - protein, 77 aa, Homo sapiens
MNLRLCVQALLLLWLSLTAVCGGSLMPLPDGNGLEDGNVRHLVQPRGSRNGPGPWQGGRRKFRRQRPRLSHKGPMPF

SEQ ID NO: 2 - protein, 12 aa, Homo sapiens
RPRLSHKGPMPF

SEQ ID NO: 3 - protein, 13 aa, Homo sapiens
QRPRLSHKGPMPF

SEQ ID NO: 4 - protein, 13 aa, Homo sapiens
  VARIANT 1: Xaa = Pyroglutamate
XRPRLSHKGPMPF

SEQ ID NO: 5 - protein, 17 aa, Homo sapiens
KFRRQRPRLSHKGPMPF

SEQ ID NO: 6 - protein, 19 aa, Homo sapiens
RRKFRRQRPRLSHKGPMPF

SEQ ID NO: 7 - protein, 36 aa, Homo sapiens
LVQPRGSRNGPGPWQGGRRKFRRQRPRLSHKGPMPF

(19) United States
(12) Patent Application Publication
(10) Pub. No.: US 2022/0096380 A1
(43) Pub. Date: Mar. 31, 2022

(54) ORAL FORMULATIONS OF A BIOLOGICALLY ACTIVE PEPTIDE AND USES THEREOF

(71) Applicant: Avive, Inc., Sunnyvale, CA (US)

(72) Inventor: Jayakumar RAJADAS, Cupertino, CA (US)

(21) Appl. No.: 17/428,591

(22) PCT Filed: Feb. 5, 2020
(86) PCT No.: PCT/US2020/016867

§ 371 Date: Aug. 4, 2021

Related U.S. Application Data
(60) Provisional application No. 62/801,250, filed on Feb. 5, 2019.

(51) Int. Cl. A61K 9/127; A61K 38/17; A61K 9/14; A61K 9/51

(52) U.S. Cl. CPC A61K 9/127; A61K 9/5161; A61K 9/146; A61K 38/1709

(57) ABSTRACT

Oral formulations comprising a biologically active peptide, such as an apel in peptide, wherein the peptide is encapsulated in particles comprising phospholipids such as 1,2-distearoyl-sn-glycero-3-phosphocholine (DSPC) and 2-dipalmitoyl-sn-glycero-3-phosphocholine (DPPC), and a poloxamer are provided. Said nanoparticles may be embedded in a carbohydrate matrix comprising a polysaccharide such as pectin, and a cross-linking agent such as calcium chloride. The nanoparticle formulation may further comprise a polyethylene glycol (PEG) and/or cholesterol. Also provided are methods of making said formulations, oral dosage forms comprising the same, and methods of treating or preventing diseases using said formulations.

### CROSS-REFERENCE TO RELATED APPLICATIONS

This application claims priority benefit to U.S. Provisional Patent Application No. 62/801,250, filed on Feb. 5, 2019, the disclosure of which is hereby incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 185632000440SEQLIST.TXT, date recorded: Feb. 4, 2020, size: 3 KB).

### TECHNICAL FIELD

The present disclosure is directed to oral formulations comprising a biologically active peptide. Also provided are oral dosage forms, methods of making, and methods of use thereof.

### BACKGROUND

Oral delivery of therapeutic polypeptides, such as peptides and proteins, is very challenging. In addition to mechanical forces exerted on orally administered compositions, polypeptides and/or liposomes degrade when subjected to the highly acidic environment of the stomach and proteases in the gastrointestinal (GI) tract. Even if polypeptides are delivered to the portion of the GI tract capable of polypeptide absorption, polypeptides (which often contain both hydrophilic and hydrophobic aspects) struggle to cross the mucus gel layer and intestinal epithelium. See, e.g., P. Shields, Drug Discover World, Fall, 2017. The result of the oral delivery of a therapeutic polypeptide is either extremely low or no bioavailability. For example, the FDA label of RYBELSUS® (an oral use semaglutide tablet) reports a bioavailability of approximately 0.4% to 1% (Reference ID: 4494169; revised 09/2019). Due to these challenges, oral delivery of therapeutic peptides is generally not seen as a viable administration route. Unfortunately, other therapeutic polypeptide administration protocols, such as injection, suffer from poor patience compliance. Thus, there exists a need in the art for oral formulations of biologically active peptides that allow for increased bioavailability of said biologically active peptides.

All references cited herein, including patent applications and publications, are incorporated by reference in their entirety.

### BRIEF SUMMARY

In one aspect, provided herein is an oral formulation of a biologically active peptide comprising a plurality of particles, wherein each particle comprises a carbohydrate matrix comprising a polysaccharide, a cross-linking agent, and a plurality of lipid-based nanoparticles embedded in the carbohydrate matrix, and wherein the lipid-based nanoparticle comprises the biologically active peptide, a poloxamer, 1,2-distearoyl-sn-glycero-3-phosphocholine (DSPC), and 2-dipalmitoyl-sn-glycero-3-phosphocholine (DPPC).

In some embodiments, the biologically active peptide comprises a stretch of at least about 15 contiguous amino acids having a net hydrophobic characteristic. In some embodiments, the biologically active peptide comprises a stretch of at least about 10 contiguous amino acids having a net positive charge at pH 7. In some embodiments, the biologically active peptide comprises, from N- to C-terminus, the stretch of amino acids having a net hydrophobic characteristic and the stretch of amino acids having a net positive charge.

In some embodiments, the lipid-based nanoparticles are liposomes comprising a lipid bilayer encapsulating a liquid core. In some embodiments, each liposome comprises a plurality of the biologically active peptide, wherein a first subset of the plurality of the biologically active peptide is configured such that one portion of the biologically active peptide is embedded in the lipid bilayer and another portion of the biologically active peptide is presented on the outer surface of the lipid bilayer or the inner surface of the lipid bilayer facing the liquid core, wherein the portion of the biologically active peptide embedded in the lipid bilayer is the stretch of amino acids having a net hydrophobic characteristic, and wherein the portion of the biologically active peptide presented on the outer surface of the lipid bilayer or the inner surface of the lipid bilayer facing the liquid core is the stretch of amino acids having a net positive charge. In some embodiments, the liquid core comprises a second subset of the plurality of the biologically active peptide.

In some embodiments, the biologically active peptide is an apelin peptide. In some embodiments, the apelin peptide is selected from the group consisting of apelin-12, apelin-13, pyroglutamyl apelin-13 ([Pyrl]-apelin-13]), apelin-17, apelin-19, and apelin-36. In some embodiments, the weight percentage of the biologically active peptide in the lipid-based nanoparticles is about 15% to about 60%.

In some embodiments, the poloxamer is poloxamer 188, poloxamer 124, poloxamer 181, poloxamer 184, poloxamer 331, and poloxamer 407, or any combination thereof. In some embodiments, the weight percentage of the poloxamer in the lipid-based nanoparticles is about 1% to about 20%.

In some embodiments, the weight percentage of DSPC in the lipid-based nanoparticles is about 5% to about 30%.

In some embodiments, the weight percentage of DPPC in the lipid-based nanoparticles is about 5% to about 30%.

In some embodiments, the lipid-based nanoparticles described herein further comprise a polyethylene glycol (PEG). In some embodiments, the average molecular weight of the PEG is about 200 Da to about 20000 Da. In some embodiments, the average molecular weight of the PEG is about 8000 Da. In some embodiments, the weight percentage of the PEG in the lipid-based nanoparticles is about 10% to about 20%.

In some embodiments, the lipid-based nanoparticles described herein further comprise cholesterol. In some embodiments, the weight percentage of cholesterol in the lipid-based nanoparticles is about 0.1% to about 10%.

In some embodiments, the lipid-based nanoparticles described herein further comprises at least one additional therapeutic agent.

In some embodiments, the lipid-based nanoparticle comprises a weight percentage of the apelin peptide of about 25%, a weight percentage of poloxamer 188 of about 8.3%, a weight percentage of DSPC of about 25%, a weight percentage of DPPC of about 25%, and a weight percentage of PEG 8000 of about 16.7%.

In some embodiments, the lipid-based nanoparticle comprises a weight percentage of the apelin peptide of about 45%, a weight percentage of poloxamer 188 of about 15%, a weight percentage of DSPC of about 10%, a weight percentage of DPPC of about 10%, a weight percentage of PEG 8000 of about 15%, and weight percentage of cholesterol of about 5%.

In some embodiments, the weight percentages of the non-solvent components in the carbohydrate matrix comprising the polysaccharide, the cross-linking agent, and the lipid-based nanoparticles is as follows: the carbohydrate matrix comprising the polysaccharide is about 48% to about 98%, the cross-linking agent is about 1% to about 5%, and the lipid-based nanoparticle is about 1% to 49%.

In some embodiments, the size range of the plurality of particles is about 1 μm to about 40 μm. In some embodiments, each of the plurality of particles comprises a plurality of pores.

In some embodiments, the polysaccharide is a pectin, gara gum, oak milk carbohydrate, or banana carbohydrate. In some embodiments, the pectin is a citrus peel pectin. In some embodiments, the pectin is 150-grade pectin.

In some embodiments, the cross-linking agent is selected from a divalent or polyvalent cation. In some embodiments, the divalent or polyvalent cation is selected from Ca²⁺, Zn²⁺, Pb²⁺, Cu²⁺, Ba²⁺, Sr²⁺, Cd⁺², Co²⁺, Ni²⁺, or a combination thereof.

In some embodiments, the biologically active peptide has a bioavailability in an individual of about 2% or greater.

In some embodiments, the plurality of particles is not a gel or hydrogel.

In another aspect, provided herein is an oral dosage form comprising any oral formulation described herein. In some embodiments, the oral dosage form comprises about 0.1 mg to about 0.5 mg of the biologically active peptide.

In some embodiments, the oral dosage form further comprises an acceptable excipient.

In some embodiments, the oral dosage form is a tablet, capsule, or caplet.

In another aspect, provided herein is a method of treating and/or preventing a disease in an individual, the method comprising administering to an individual any oral dosage form described herein.

In another aspect, provided herein is a method of making any oral formulation described herein, the method comprising admixing the carbohydrate matrix comprising the polysaccharide, the cross-linking agent, the biologically active peptide, the poloxamer, DSPC, and DPPC, thereby obtaining the oral formulation. In some embodiments, the method further comprises admixing the PEG and/or cholesterol with the carbohydrate matrix comprising the polysaccharide, the cross-linking agent, the biologically active peptide, the poloxamer, DSPC, and DPPC.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### DETAILED DESCRIPTION

Provided herein, in some aspects, is an oral formulation of a biologically active peptide comprising a plurality of particles, wherein each particle comprises a carbohydrate matrix comprising a polysaccharide, a cross-linking agent, and a plurality of lipid-based nanoparticles embedded in the carbohydrate matrix, and wherein the lipid-based nanoparticles comprise the biologically active peptide. The work described herein involves development of oral formulations for peptides that are currently considered as not-suitable for oral formulations. The present disclosure is based, in part, on the inventor's unique insights for an oral formulation of a biologically active peptide that resists peptide degradation due to the highly acidic environment of the stomach and GI tract proteases. The oral formulations are designed to release the therapeutic molecule in the intestine, and present the biologically active peptide for absorption in the intestine. Additionally, the biologically active peptide, which is presented for absorption via lipid-based nanoparticles, evades first pass metabolism of the liver. The result is an oral formulation that provides enhanced bioavailability for biologically active peptides.

Also provided herein, in some aspects, are oral dosage forms comprising the oral formulations described herein, methods of making the oral formulations described herein, and methods of use, such as methods of treating and/or preventing a disease in an individual, using the oral dosage forms and oral formulations described herein.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### Definitions

For purposes of interpreting this specification, the following definitions will apply and, whenever appropriate, terms used in the singular will also include the plural and vice versa. In the event that any definition set forth below conflicts with any document incorporated herein by reference, the definition set forth shall control.

The term “peptide,” such as used in the phrase “biologically active peptide,” refers to a polymer comprising amino acid residues, and is not to be construed as implying a limitation regarding the number of amino acids and/or length thereof. Such polymers may contain natural amino acids and/or or non-natural amino acid. In some embodiments, the term “polypeptide” also encompasses modified species of polypeptides, e.g., polypeptides comprising one or more chemical modifications and/or one or more post-translational modifications.

The term “sequence identity,” with respect to a polypeptide or peptide comprising an amino acid sequence refers to the percentage of amino acid residues in a candidate sequence that are identical to the amino acid residues in the specific protein or amino acid sequence after aligning the sequences and introducing gaps, if necessary, to achieve a maximum percent sequence identity, and not considering any conservative substitutions as part of the sequence identity. Alignment can be achieved by any method known to one of skill in the art, for example, by using publicly available programs such as BLAST and EMBOSS. Those skilled in the art can determine appropriate parameters for measuring alignment, including any algorithms needed to achieve maximal alignment over the full length of the sequences being compared.

As used herein, the terms “treating” or “preventing,” or grammatical equivalents thereof, encompass approaches for obtaining or maintaining beneficial or desired results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the disease, preventing or delaying the spread of the disease, preventing or delaying the recurrence of the disease, delaying or slowing the progression of the disease, ameliorating the disease state, providing a remission (e.g., partial or total) of the disease, delaying the progression of the disease, increasing the quality of life, and/or prolonging survival. The methods of the present application contemplate any one or more of these aspects of treatment.

The term “individual” refers to a mammal and includes, but is not limited to, human, bovine, horse, feline, canine, rodent, or primate.

The term “pharmaceutically acceptable,” as used herein, is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers, excipients, or salts have preferably met the required standards of toxicological and manufacturing testing and/or are included on the Inactive Ingredient Guide prepared by the U.S. Food and Drug administration.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.” In some embodiments, numerical designations are provided herein for ease of understanding the scope of the present disclosure, wherein the numerical designations are calculated from experimental values and may include approximations, e.g., rounded weight percentages calculated from an amount of a starting material. In some embodiments, numerical designations provided herein, e.g., weight percentages, may vary (±) by increments of 0.1 to 0.5.

As used herein, including in the appended claims, the singular forms “a,” “or,” and “the” include plural referents unless the context clearly dictates otherwise.

### A. Oral Formulations of a Biologically Active Peptide

The present application provides, in some aspects, an oral formulation of a biologically active peptide comprising a plurality of particles, wherein each particle comprises a carbohydrate matrix comprising a polysaccharide, a cross-linking agent, and a plurality of lipid-based nanoparticles embedded in the carbohydrate matrix, and wherein the lipid-based nanoparticles comprise the biologically active peptide. In some embodiments, each of the plurality of lipid-based nanoparticles are not individually encapsulated by the carbohydrate matrix. In some embodiments, one or more of the lipid-based nanoparticles are not completely encapsulated by the carbohydrate matrix. In some embodiments, the carbohydrate matrix is not a surface coating on a lipid-based nanoparticle.

The oral formulations described herein encompass a range of working component weight percentages. One of ordinary skill in the art will readily recognize that descriptions using weight percentages are based on the components included in the total weight used in the weight percentage calculation. For example, adding and/or subtracting one or more additional components to an oral formulations described herein will adjust the weight percentages of the other components of the oral formulation if included in the total weight used in the weight percentage calculation. Thus, in some embodiments, weight percentages are provided relative to a list of one or more provided components used to calculate the total weight used in the weight percentage calculation. In some embodiments, the oral formulation comprises a carbohydrate matrix comprising a polysaccharide, a cross-linking agent, and a plurality of lipid-based nanoparticles comprising a plurality of lipid-based nanoparticles comprising a biologically active peptide, a poloxamer, 1,2-distearoyl-sn-glycero-3-phosphocholine (DSPC), 2-dipalmitoyl-sn-glycero-3-phosphocholine (DPPC), and optionally a PEG and/or cholesterol (included in the weight percentage calculation when present), wherein: (i) the weight percentage of the carbohydrate matrix comprising the polysaccharide relative to the polysaccharide, the cross-linking agent, the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about 48% to about 98%; (ii) the weight percentage of the cross-linking agent relative to the polysaccharide, the cross-linking agent, the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about 1% to about 5%; and (iii) the weight percentage of the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), of the lipid-based nanoparticles relative to the polysaccharide, the cross-linking agent, the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about 1% to 49%. In some instances of any of the embodiments provided herein, the weight percentage of the carbohydrate matrix comprising the polysaccharide is larger than the weight percentage of the lipid-based nanoparticles.

In some embodiments, the weight percentage of the carbohydrate matrix comprising the polysaccharide relative to the polysaccharide, the cross-linking agent, the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about any of 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98%.

In some embodiments, the weight percentage of the cross-linking agent relative to the polysaccharide, the cross-linking agent, the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about any of 1%, 2%, 3%, 4%, or 5%.

In some embodiments, the weight percentage of the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), of the lipid-based nanoparticles relative to the polysaccharide, the cross-linking agent, the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about any of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49%.

In some embodiments, the total amount a biologically active peptide in an oral formulation described herein is based on the amount of a lipid-based nanoparticle relative to a carbohydrate matrix comprising a polysaccharide and a cross-linking agent. For example, in some embodiments, an oral formulation having a relatively low amount of a biologically active peptide comprises weight percentages of the non-solvent components in the carbohydrate matrix comprising the polysaccharide, the cross-linking agent, and the lipid-based nanoparticles as follows: the weight percentage of the carbohydrate matrix comprising the polysaccharide is about 98%, the weight percentage of the cross-linking agent is about 1%, and the weight percentage of the lipid-based nanoparticle is about 1%. In some embodiments, an oral formulation having a relatively high amount of a biologically active peptide comprises weight percentages of the non-solvent components in the carbohydrate matrix comprising the polysaccharide, the cross-linking agent, and the lipid-based nanoparticles as follows: the weight percentage of the carbohydrate matrix comprising the polysaccharide is about 50%, the weight percentage of the cross-linking agent is about 1%, and the weight percentage of the lipid-based nanoparticle is about 49%. In some embodiments, an oral formulation having a relatively high amount of a biologically active peptide and a cross-linking agent comprises weight percentages of the non-solvent components in the carbohydrate matrix comprising the polysaccharide, the cross-linking agent, and the lipid-based nanoparticles as follows: the weight percentage of the carbohydrate matrix comprising the polysaccharide is about 48%, the weight percentage of the cross-linking agent is about 5%, and the weight percentage of the lipid-based nanoparticle is about 47%. In some embodiments, the weight percentage of the carbohydrate matrix comprising the polysaccharide is larger than the weight percentage of the lipid-based nanoparticles.

In some aspects, provided herein is an oral formulation described herein produced using a spraying, such as spray-drying, technique or a microemulsion technique described herein.

The oral formulations described herein provide enhanced bioavailability of the biologically active peptide therein, such as compared to when the biologically active peptide is administered in any of the following ways: alone, in a lipid-based nanoparticle not embedded in a carbohydrate matrix comprising a polysaccharide, and in a carbohydrate matrix comprising a polysaccharide without a lipid-based nanoparticle. In some embodiments, when the oral formulation is administered to an individual, such as a human, the biologically active peptide has a bioavailability in an individual of about 1% or greater, such as about any of 1.1% or greater, 1.2% or greater, 1.3% or greater, 1.4% or greater, 1.5% or greater, 1.6% or greater, 1.7% or greater, 1.8% or greater, 1.9% or greater, 2% or greater, 2.1% or greater, 2.2% or greater, 2.3% or greater, 2.4% or greater, 2.5% or greater, 2.6% or greater, 2.7% or greater, 2.8% or greater, 2.9% or greater, 3% or greater, 3.1% or greater, 3.2% or greater, 3.3% or greater, 3.4% or greater, 3.5% or greater, 3.6% or greater, 3.7% or greater, 3.8% or greater, 3.9% or greater, 4% or greater, 4.1% or greater, 4.2% or greater, 4.3% or greater, 4.4% or greater, 4.5% or greater, 4.6% or greater, 4.7% or greater, 4.8% or greater, 4.9% or greater, 5% or greater, 6% or greater, 7% or greater, 8% or greater, 9% or greater, or 10% or greater.

In some embodiments, the oral formulation is in a state that maintains the structure of the components described herein, e.g., a particle comprising a carbohydrate matrix comprising a polysaccharide, a cross-linking agent, and a plurality of lipid-based nanoparticles embedded in the carbohydrate matrix. In some embodiments, the oral formulation is in a state that is suitable for oral administration. In some embodiments, the oral formulation is in a state suitable for use in an oral dosage form. In some embodiments, the oral formulation is a dried formulation, such as a dried powder.

In some embodiments, the plurality of particles further comprises at least one additional therapeutic agent described herein.

i. Lipid-Based Nanoparticles and Components Thereof

The oral formulations described herein comprise a plurality of lipid-based nanoparticles embedded in a carbohydrate matrix comprising a polysaccharide. In some embodiments, the lipid-based nanoparticles comprise a biologically active peptide, a poloxamer, 1,2-distearoyl-sn-glycero-3-phosphocholine (DSPC), and 2-dipalmitoyl-sn-glycero-3-phosphocholine (DPPC). In some embodiments, the lipid-based nanoparticles comprise a polyethylene glycol (PEG) and/or cholesterol. In some embodiments, the lipid-based nanoparticle is a liposome.

a. Biologically Active Peptides and Configurations Thereof.

In some aspects, the biologically active peptide, or portion(s) thereof, described herein are designed and/or selected such that a first portion of the biologically active peptide is embedded in the lipid-based nanoparticle and a second portion of the biologically active peptide is associated with a surface of the lipid-based nanoparticle, such as the outer or inner surface of a lipid bilayer.

In some embodiments, the biologically active peptide comprises a stretch of at least about 15, such as at least about any of 20, 25, or 30, contiguous amino acids having a net hydrophobic characteristic. In some embodiments, the biologically active peptide comprises a stretch of at least about 15, such as at least about any of 20, 25, or 30, contiguous amino acids, wherein the stretch comprises more hydrophobic amino acid residues than hydrophilic amino acid residues. In some embodiments, the biologically active peptide comprises a stretch of at least about 15, such as at least about any of 20, 25, or 30, contiguous amino acids, wherein the stretch comprises at least about 55%, such as at least about any of 60%, 65%, 70%, 75%, 80%, 85%, 90%, 95%, or 100%, hydrophobic amino acid residues. One of ordinary skill in the art will readily understand and be able to identify hydrophobic amino acids, e.g., glycine, alanine, valine, leucine, isoleucine, proline, phenylalanine, methionine, and tryptophan.

In some embodiments, the biologically active peptide comprises a stretch of at least about 10, such as at least about any of 15, 20, 25, or 30, contiguous amino acids having a net positive charge at pH 7. In some embodiments, the biologically active peptide comprises a stretch of at least about 10, such as at least about any of 15, 20, 25, or 30, contiguous amino acids having a net positive charge at a physiological pH of the gastrointestinal tract. One of ordinary skill in the art will readily understand and be able to identify charged amino acids and the impact of pH on the charge of an amino acid, e.g., lysine and arginine.

In some embodiments, the biologically active peptide comprises a stretch of at least about 15, such as at least about any of 20, 25, or 30, contiguous amino acids having a net hydrophobic characteristic at or near, such as within about 5 amino acids, the N-terminus of the biologically active peptide. In some embodiments, the biologically active peptide comprises a stretch of at least about 10, such as at least about any of 15, 20, 25, or 30, contiguous amino acids having a net positive charge at pH 7 at or near, such as within about 5 amino acids, of the C-terminus. In some embodiments, the biologically active peptide comprises, from N- to C-terminus, the stretch of amino acids having a net hydrophobic characteristic and the stretch of amino acids having a net positive charge. In some embodiments, the biologically active peptide comprises one or more stretches of other amino acids between the stretch of amino acids having a net hydrophobic characteristic and the stretch of amino acids having a net positive charge. In some embodiments, the biologically active peptide comprises, from N- to C-terminus, a stretch of amino acids having a net hydrophobic characteristic, a stretch of at least about 5, such as at least about any of 10, 15, 20, 25, 30, 35, or 40, amino acids, and a stretch of amino acids having a net positive charge.

In some embodiments, the lipid-based nanoparticles are liposomes comprising a lipid bilayer encapsulating a liquid core. In some embodiments, wherein each liposome comprises a plurality of the biologically active peptide, a first subset of the plurality of the biologically active peptide is configured such that one portion of the biologically active peptide is embedded in the lipid bilayer and another portion of the biologically active peptide is presented on the outer surface of the lipid bilayer or the inner surface of the lipid bilayer facing the liquid core, wherein the portion of the biologically active peptide embedded in the lipid bilayer is the stretch of amino acids having a net hydrophobic characteristic, and wherein the portion of the biologically active peptide presented on the outer surface of the lipid bilayer or the inner surface of the lipid bilayer facing the liquid core is the stretch of amino acids having a net positive charge. In some embodiments, the lipid-based nanoparticle, such as a liposome, is configured such that a biologically active peptide presented on the outer surface of the lipid-based nanoparticle may associate with, such as bind, a relevant receptor and/or target binding site.

In some embodiments, the lipid-based nanoparticle comprises a liquid core comprising a second subset of the plurality of the biologically active peptide. In some embodiments, the lipid-based nanoparticle, such as a liposome, is configured to hold a certain concentration, or range thereof, of the second subset of the biologically active peptide.

In some embodiments, the biologically active peptide is an apelin peptide. In some embodiments, the apelin peptide is selected from the group consisting of apelin-12, apelin-13, pyroglutamyl apelin-13 ([Pyrl]-apelin-13]), apelin-17, apelin-19, and apelin-36. In some embodiments, the apelin peptide is pyroglutamyl apelin-13 ([Pyrl]-apelin-13]).

Apelin peptides, and biologically active variants, within the scope of the present disclosure are described in U.S. PG Patent Publication. No. 2016/0058705, which is incorporated herein by reference in its entirety. In some embodiments, the apelin peptide comprises an amino acid sequence selected from the group consisting of SEQ ID NOS:1-7 (Table 1). In some embodiments, the apelin peptide comprises a sequence having at least about any of 60%, 65%, 70%, 75%, 80%, 85%, 90%, 95%, 96%, 97%, 98%, or 99% sequence identity to a sequence from SEQ ID NOS:1-7. In some embodiments, the apelin peptide, such as any apelin peptide from SEQ ID NOS:1-7, comprises one or more, such as any of 2, 3, 4, or 5, amino acid changes selected from any one or more of an addition, substitution, and/or deletion. In some embodiments, the apelin peptide comprises a modification, such as a post-translation modification.

TABLE 1
Apelin peptide sequences.

| SEQ | | |
|---|---|---|
| ID | | |
| NO. | Sequence | Name |
| 1 | Met Asn Leu Arg Leu Cys Val Gln Ala | Apelin |
| | Leu Leu Leu Leu Trp Leu Ser Leu Thr | pre- |
| | Ala Val Cys Gly Gly Ser Leu Met Pro | protein |
| | Leu Pro Asp Gly Asn Gly Leu Glu Asp | |
| | Gly Asn Val Arg His Leu Val Gln Pro | |
| | Arg Gly Ser Arg Asn Gly Pro Gly Pro | |
| | Trp Gln Gly Gly Arg Arg Lys Phe Arg | |
| | Arg Gln Arg Pro Arg Leu Ser His Lys | |
| | Gly Pro Met Pro Phe | |
| 2 | Arg Pro Arg Leu Ser His Lys Gly Pro | Apelin- |
| | Met Pro Phe | 12 |
| 3 | Gln Arg Pro Arg Leu Ser His Lys Gly | Apelin- |
| | Pro Met Pro Phe | 13 |
| 4 | Xaa Arg Pro Arg Leu Ser His Lys Gly | [Pyr1]- |
| | Pro Met Pro Phe | apelin- |
| | (Xaa/X is pyroglutamate) | 13 |
| 5 | Lys Phe Arg Arg Gln Arg Pro Arg Leu | Apelin- |
| | Ser His Lys Gly Pro Met Pro Phe | 17 |
| 6 | Arg Arg Lys Phe Arg Arg Gln Arg Pro | Apelin- |
| | Arg Leu Ser His Lys Gly Pro Met Pro | 19 |
| | Phe | |
| 7 | Leu Val Gln Pro Arg Gly Ser Arg Asn | Apelin- |
| | Gly Pro Gly Pro Trp Gln Gly Gly Arg | 36 |
| | Arg Lys Phe Arg Arg Gln Arg Pro Arg | |
| | Leu Ser His Lys Gly Pro Met Pro Phe | |

In some embodiments, the biologically active peptide is a GHK peptide, a KRDS peptide, or a biotin-KRDS peptide.

In some embodiments, the weight percentage of the biologically active peptide (e.g., the apelin peptide) in the lipid-based nanoparticles is between about 1% and about 70%, such as between any of about 5% and about 60%, about 15% and about 60%, about 15% and about 35%, about 20% and about 30%, about 22.5% and about 27.5%, about 24% and about 26%, about 35% and about 55%, about 40% and about 50%, about 42.5% and about 47.5%, or about 44% and about 46%. In some embodiments, the weight percentage of the biologically active peptide (e.g., the apelin peptide) in the lipid-based nanoparticles is at least about 15%, such as at least about any of 20%, 25%, 30%, 35%, 40%, 45%, 50%, 55%, 60%, or 65%. In some embodiments, the weight percentage of the biologically active peptide (e.g., the apelin peptide) in the lipid-based nanoparticles is about 70% or less, such as about any of 65% or less, 60% or less, 55% or less, 50% or less, 45% or less, 40% or less, 35% or less, 30% or less, 25% or less, 20% or less, 15% or less, or 10% or less. In some embodiments, the weight percentage of the biologically active peptide (e.g., the apelin peptide) in the lipid-based nanoparticles is about any of 5%, 10%, 15%, 20%, 25%, 30%, 35%, 40%, 45%, 50%, 55%, 60%, 65%, or 70%.

b. Other Components of the Lipid-Based Nanoparticles

The lipid-based nanoparticles described herein comprise a poloxamer. In some embodiments, the poloxamer is poloxamer 188, poloxamer 124, poloxamer 181, poloxamer 184, poloxamer 331, and poloxamer 407, or any combination thereof. In some embodiments, the poloxamer is poloxamer 188.

In some embodiments, the weight percentage of the poloxamer in the lipid-based nanoparticles is between about 1% and about 25%, such as between any of about 1% and about 20%, about 2% and about 14%, about 5% and about 11%, about 8% and about 9%, about 7.3% and about 9.3%, about 10% and about 20%, about 17.5% and about 22.5%, or about 14% and about 16%. In some embodiments, the weight percentage of the poloxamer in the lipid-based nanoparticles is at least about 1%, such as at least about any of 2%, 3%, 4%, 5%, 6%, 7%, 8%, 9%, 10%, 11%, 12%, 13%, 14%, 15%, 16%, 17%, 18%, 19%, or 20%. In some embodiments, the weight percentage of the poloxamer in the lipid-based nanoparticles is about 20% or less, such as about any of 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, or 2% or less. In some embodiments, the weight percentage of the poloxamer in the lipid-based nanoparticles is about any of 1%, 2%, 3%, 4%, 5%, 6%, 7%, 8%, 8.3%, 9%, 10%, 11%, 12%, 13%, 14%, 15%, 16%, 17%, 18%, 19%, 20%, 21%, 22%, 23%, 24%, or 25%.

The lipid-based nanoparticles described herein comprise 1,2-distearoyl-sn-glycero-3-phosphocholine (DSPC). In some embodiments, the weight percentage of DSPC in the lipid-based nanoparticles is between about 5% and about 30%, such as between any of 5% and about 15%, about 7.5% and about 12.5%, about 9% and about 11%, about 20% and about 30%, about 22.5% and about 27.5%, or about 24% and about 26%.

In some embodiments, the weight percentage of DSPC in the lipid-based nanoparticles is at least about 5%, such as at least about any of 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the weight percentage of DSPC in the lipid-based nanoparticles is about 30% or less, such as about any of 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less. In some embodiments, the weight percentage of DSPC in the lipid-based nanoparticles is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

The lipid-based nanoparticles described herein comprise 2-dipalmitoyl-sn-glycero-3-phosphocholine (DPPC). In some embodiments, the weight percentage of DPPC in the lipid-based nanoparticles is between about 5% and about 30%, such as between any of 5% and about 15%, about 7.5% and about 12.5%, about 9% and about 11%, about 20% and about 30%, about 22.5% and about 27.5%, or about 24% and about 26%.

In some embodiments, the weight percentage of DPPC in the lipid-based nanoparticles is at least about 5%, such as at least about any of 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the weight percentage of DPPC in the lipid-based nanoparticles is about 30% or less, such as about any of 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less. In some embodiments, the weight percentage of DPPC in the lipid-based nanoparticles is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

In some embodiments, the lipid-based nanoparticles described herein comprise a PEG. In some embodiments, the PEG has an average molecular weight of between about 200 to about 20,000 Daltons. In some embodiments, the PEG is PEG 200, PEG 300, PEG 400, PEG 1000, PEG 1540, PEG 4000, PEG 5000, PEG 6000, PEG 7000, PEG 8000, PEG 9000, or PEG 10000. In some embodiments, the PEG is PEG 8000.

In some embodiments, the weight percentage of the PEG in the lipid-based nanoparticles is between about 10% and about 20%, such as between any of about 12.5% and about 17.5%, about 14% and about 16%, about 15.6% and about 17.6%. In some embodiments, the weight percentage of the PEG in the lipid-based nanoparticles is at least about 10%, such as at least about any of 11%, 12%, 13%, 14%, 15%, 16%, 17%, 18%, 19%, or 20%. In some embodiments, the weight percentage of the PEG in the lipid-based nanoparticles is about 20% or less, such as about any of 19% or less, 18% or less, 17% or less, 16% or less, 15% or less, 14% or less, 13% or less, 12% or less, 11% or less, or 10% or less. In some embodiments, the weight percentage of the PEG in the lipid-based nanoparticles is about any of 10%, 11%, 12%, 13%, 14%, 15%, 16%, 16.6%, 17%, 18%, 19%, or 20%.

In some embodiments, the lipid-based nanoparticles described herein comprise cholesterol. In some embodiments, the weight percentage of cholesterol in the lipid-based nanoparticles is between about 0.1% and about 10%, such as between any of about 2.5% and about 7.5%, or about 4% and about 6%.

In some embodiments, the weight percentage of cholesterol in the lipid-based nanoparticles is at least about 0.1%, such as at least about any of 1%, 2%, 3%, 4%, 5%, 6%, 7%, 8%, 9%, or 10%. In some embodiments, the weight percentage of cholesterol in the lipid-based nanoparticles is about 10% or less, such as about any of 9% or less, 8% or less, 7% or less, 6% or less, 5% or less, 4% or less, 3% or less, 2% or less, or 1% or less. In some embodiments, the weight percentage of cholesterol in the lipid-based nanoparticles is about any of 1%, 2%, 3%, 4%, 5%, 6%, 7%, 8%, 9%, or 10%.

In some embodiments, the lipid-based nanoparticle comprises a weight percentage of the apelin peptide of between about 23% and 27%, a weight percentage of the poloxamer (e.g., poloxamer 188) of between about 6.3% and 10.3%, a weight percentage of DSPC of between about 23% and about 27%, a weight percentage of DPPC of between about 23% and about 27%, and a weight percentage of the PEG (e.g., PEG 8000) of between about 14.7% and about 18.7%.

In some embodiments, the lipid-based nanoparticle comprises a weight percentage of the apelin peptide of about 25%, a weight percentage of poloxamer 188 of about 8.3%, a weight percentage of DSPC of about 25%, a weight percentage of DPPC of about 25%, and a weight percentage of PEG 8000 of about 16.7%.

In some embodiments, the lipid-based nanoparticle comprises a weight percentage of the apelin peptide of between about 43% and 47%, a weight percentage of the poloxamer (e.g., poloxamer 188) of between about 13% and about 17%, a weight percentage of DSPC of between about 8% and about 12%, a weight percentage of DPPC of between about 8% and about 12%, a weight percentage of the PEG (e.g., PEG 8000) of between about 13% and about 17%, and weight percentage of cholesterol of between about 3% and about 7%.

In some embodiments, the lipid-based nanoparticle comprises a weight percentage of the apelin peptide of about 45%, a weight percentage of poloxamer 188 of about 15%, a weight percentage of DSPC of about 10%, a weight percentage of DPPC of about 10%, a weight percentage of PEG 8000 of about 15%, and weight percentage of cholesterol of about 5%.

In some embodiments, the lipid-based nanoparticle is a liposome prepared according to Formulation 1 (Table 2). In some embodiments, the lipid-based nanoparticle is a liposome prepared according to Formulation 2 (Table 2).

TABLE 2
Formulation 1 and Formulation 2.

| | Formulation 1 | | Formulation 2 | |
|---|---|---|---|---|
| | Weight (mg) | Weight % | Weight (mg) | Weight % |
| DSPC | 450 | 25.00% | 100 | 10.00% |
| DPPC | 450 | 25.00% | 100 | 10.00% |
| Poloxamer 188 | 150 | 8.33% | 150 | 15.00% |
| PEG 8000 | 300 | 16.67% | 150 | 15.00% |
| [Pyr1]-Apelin-13 | 450 | 25.00% | 450 | 45.00% |
| Cholesterol | — | — | 50 | 5.00% |
| TOTAL | 1800 | 100.00% | 1000 | 100.00% |

Methods of making lipid-based nanoparticles, such as liposomes, comprising a biologically active peptide embedded therein are known in the art. In some embodiments, the lipid-based nanoparticles, such as liposomes, are made by admixing a poloxamer, DSPC, DPPC, and optionally a PEG and/or cholesterol, to form a lipid film. The biologically active peptide is then slowly added to the lipid film, thereby forming the lipid-based nanoparticles. See, e.g., International application publication WO2018075822, which is hereby incorporated herein in its entirety.

c. Other Therapeutic Agents

In some embodiments, the lipid-based nanoparticle further comprises at least one additional therapeutic agent. In some embodiments, the at least one additional therapeutic agent is selected from the group consisting of inotropes, beta adrenergic receptor blockers, HMG-Co-A reductase inhibitors, angiotensin II receptor antagonists, angiotensin converting enzyme (ACE) inhibitors, calcium channel blockers (CCB), endothelin antagonists, renin inhibitors, diuretics, ApoA-1 mimetics, anti-diabetic agents, obesity-reducing agents, aldosterone receptor blockers, endothelin receptor blockers, aldosterone synthase inhibitors (ASI), a CETP inhibitor, anti-coagulants, relaxin, BNP (nesiritide) and/or a NEP inhibitor. In some embodiments, the additional therapeutic agent is an ACE inhibitor, relaxin, a natriuretic peptide, ghrelin, and other bioactive peptides (such as disclosed in, e.g., WO2018075822; Erdmann, 2008; and Chakrabarti, 2016, each of which are hereby incorporated by reference herein by in its entirety). In some embodiments, the additional therapeutic agent comprises valsartan, candesartan, or losartan.

Inotropes include, for example, dobutamine, isoproterenol, milrinone, amirinone, levosimendan, epinephrine, norepinephrine, isoproterenol, and digoxin. Beta adrenergic receptor blockers include, for example, acebutolol, atenolol, betaxolol, bisoprolol, carteolol, metoprolol, nadolol, propranolol, sotalol, and timolol. Anti-coagulants include, for example, Dalteparin, Danaparoid, Enoxaparin, Heparin, Tinzaparin, and Warfarin. HMG-Co-A reductase inhibitors (also called beta-hydroxy-beta-methylglutaryl-co-enzyme-A reductase inhibitors) include active agents that may be used to lower the lipid levels including cholesterol in blood. Examples of HMG-Co-A reductase inhibitors include, for example, atorvastatin, cerivastatin, compactin, dalvastatin, dihydrocompactin, fluindostatin, fluvastatin, lovastatin, pitavastatin, mevastatin, pravastatin, rosuvastatin, rivastatin, simvastatin, velostatin, and pharmaceutically acceptable salts thereof. ACE-inhibitors (also called angiotensin converting enzyme inhibitors) include molecules that interrupt the enzymatic degradation of angiotensin Ito angiotensin II. ACE-inhibitors include compounds that may be used for the regulation of blood pressure and for the treatment of congestive heart failure. Examples of ACE-inhibitors include, for example, alacepril, benazepril, benazeprilat, captopril, ceronapril, cilazapril, delapril, enalapril, enaprilat, fosinopril, imidapril, lisinopril, moexipril, moveltopril, perindopril, quinapril, ramipril, spirapril, temocapril, trandolapril, and zofenopril, or pharmaceutically acceptable salt thereof. Endothelin antagonists include, for example, bosentan, and tezosentan, or pharmaceutically acceptable salts thereof.

ii. Carbohydrate Matrices Comprising a Polysaccharide

The oral formulations described herein comprise a plurality of particles comprising a carbohydrate matrix comprising a polysaccharide.

In some embodiments, the size range of the plurality of particles is between about 1 μm and about 40 μm, such as between any of about 1 μm and about 10 μm, about 1 μm and about 20 μm, about 1 μm and about 30 μm, about 5 μm and about 25 μm, about 5 μm and about 35 μm, about 10 μm and about 40 μm, about 20 μm and about 40 μm, about 30 μm and about 40 inn, or about 20 μm and about 30 μm. In some embodiments, the average size of the plurality of particles is at least about 1 μm, such as at least about any of 2 μm, 3 μm, 4 μm, 5 μm, 10 μm, 15 inn, 20 μm, 25 μm, 30 μm, 40 μm, 45 μm, 50 μm, 55 μm, 60 μm, 65 μm, 70 μm, 75 μm, 80 μm, 85 μm, 90 μm, 95 μm, or 100 μm. In some embodiments, the average size of the plurality of particles is about 100 μm or less, such as about any of 95 μm or less, 90 μm or less, 85 μm or less, 80 μm or less, 75 μm or less, 70 μm or less, 65 μm or less, 60 μm or less, 55 μm or less, 50 μm or less, 45 μm or less, 40 μm or less, 35 μm or less, 30 μm or less, 25 μm or less, 20 μm or less, 15 μm or less, 10 μm or less, 5 μm or less, 4 μm or less, 3 μm or less, 2 μm or less, or 1 μm or less. In some embodiments, the size of the plurality of particles is homogenous. In some embodiments, the size of the plurality of particles is heterogeneous. In some embodiments, the size of the particle is as measured by dynamic light scattering.

In some embodiments, the plurality of particles are produced via a spray drying technique and/or milling technique.

In some embodiments, each of the plurality of particles comprises a plurality of pores. In some embodiments, the porosity of each of the plurality of particles is configured to adjust the amount of a lipid-based nanoparticle embedded therein.

In some embodiments, the polysaccharide is a pectin, gara gum, oak milk carbohydrate, banana carbohydrate, or any combination thereof. In some embodiments, the polysaccharide is a pectin. In some embodiments, the pectin is a citrus peel pectin. In some embodiments, the pectin is 150-grade pectin. In some embodiments, the pectin has a degree of esterification below about any of 70%, 65%, 60%, 55%, 50%, 45%, 40%, 35%, 30%, 25%, 20%, 15%, or 10%. In some embodiments, the degree of esterification of the pectin is selected based on a desired degree of cross-linking of the plurality of particles.

In some embodiments, the plurality of particles is not a gel or hydrogel.

In some embodiments, the carbohydrate matrix further comprises at least one additional therapeutic agent. In some embodiments, the at least one additional therapeutic agent is selected from the group consisting of inotropes, beta adrenergic receptor blockers, HMG-Co-A reductase inhibitors, angiotensin II receptor antagonists, angiotensin converting enzyme (ACE) inhibitors, calcium channel blockers (CCB), endothelin antagonists, renin inhibitors, diuretics, ApoA-1 mimetics, anti-diabetic agents, obesity-reducing agents, aldosterone receptor blockers, endothelin receptor blockers, aldosterone synthase inhibitors (ASI), a CETP inhibitor, anti-coagulants, relaxin, BNP (nesiritide) and/or a NEP inhibitor. In some embodiments, the additional therapeutic agent is an ACE inhibitor, relaxin, a natriuretic peptide, ghrelin, and other bioactive peptides (such as disclosed in, e.g., WO2018075822; Erdmann, 2008; and Chakrabarti, 2016, each of which are hereby incorporated by reference herein by in its entirety). In some embodiments, the additional therapeutic agent comprises valsartan, candesartan, or losartan. In some embodiments, the carbohydrate matrix further comprises one or more of resveratrol, curcumin, and carnitine.

iii. Cross-Linking Agents

The oral formulations described herein comprise a plurality of particles comprising a cross-linking agent. In some embodiments, the cross-linking agent is a non-covalent cross-linking agent. In some embodiments, the cross-linking agent is a covalent cross-linking agent (e.g., generates one or more covalent linkages in a component, and/or between components, of the oral formulation).

In some embodiments, the cross-linking agent forms intra-particle crosslinks between portions of the carbohydrate matrix (e.g., between the polysaccharide). In some embodiments, the cross-linking agent forms intra-particle crosslinks between a portion of the carbohydrate matrix and a portion of the lipid-based nanoparticle (e.g., the biologically active peptide). In some embodiments, the cross-linking agent forms inter-particle crosslinks.

In some embodiments, the cross-linking agent is selected from a divalent or polyvalent cation. In some embodiments, the divalent or polyvalent cation is selected from Ca²⁺, Zn²⁺, Pb²⁺, Cu²⁺, Ba²⁺, Sr²⁺, Cd⁺², Co²⁺, Ni²⁺, or a combination thereof. In some embodiments, the cross-linking agent is Ca²⁺. In some embodiments, the cross-linking agent is from a composition capable of generating Ca²⁺, such as CaCl₂. In some embodiments, the cross-linking agent is Zn²⁺. In some embodiments, the cross-linking agent is from a composition capable of generating Zn²⁺, such as ZnSO₄.

### B. Oral Dosage Forms

In some aspects, provided herein are oral dosage forms comprising an oral formulation described herein. In some embodiments, the oral dosage form comprises more than one oral formulation described herein, wherein each oral formulation is unique from the others in the oral dosage form, e.g., each has a different amount of a biologically active peptide and/or a different weight percentage of the carbohydrate matrix.

In some embodiments, the oral dosage form comprises between about 0.01 mg and about 1 mg, such as between any of about 0.015 mg and about 0.1 mg, about 0.02 mg and about 0.03 mg, about 0.02 and about 0.1 mg, about 0.1 mg and about 0.5 mg, and about 0.5 mg and about 0.75 mg, of the biologically active peptide. In some embodiments, the oral dosage form comprises at least about 0.01 mg, such as at least about any of 0.025 mg, 0.05 mg, 0.075 mg, 0.1 mg, 0.2 mg, 0.3 mg, 0.4 mg, 0.5 mg, 0.6 mg, 0.7 mg, 0.8 mg, 0.9 mg, or 1 mg, of the biologically active peptide. In some embodiments, the oral dosage form comprises about any of the following amounts of the biologically active peptide: 0.01 mg, 0.025 mg, 0.05 mg, 0.075 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, or 1 mg.

In some embodiments, the oral dosage form is a tablet, capsule, or caplet. In some embodiments, the oral dosage form comprises a vegetable- or gelatin-based capsule. In some embodiments, the oral dosage form comprises an oral formulation in a state suitable for oral administration. In some embodiments, the oral formulation in the oral dosage form is in a dried form, a semi-liquid form (such as a gel), or a liquid form (such as a suspension, solution, or emulsion).

In some embodiments, the oral dosage form further comprises a pharmaceutically acceptable excipient, pharmaceutically acceptable salt, diluent, carrier, vehicle, bulking agent, other inactive agents used to formulate oral dosage forms, or any combination thereof. Vehicles and excipients commonly employed in oral dosage forms include, for example, talc, gum Arabic, lactose, starch, magnesium stearate, cocoa butter, and paraffin derivatives. In some embodiments, the oral dosage form further comprises a preservative and/or a stabilizer. In some embodiments, the oral dosage form further comprises a cryoprotectant agent.

In some embodiments, the oral drug dosage form further comprises at least one additional therapeutic agent. In some embodiments, the at least one additional therapeutic agent is selected from the group consisting of inotropes, beta adrenergic receptor blockers, HMG-Co-A reductase inhibitors, angiotensin II receptor antagonists, angiotensin converting enzyme (ACE) inhibitors, calcium channel blockers (CCB), endothelin antagonists, renin inhibitors, diuretics, ApoA-1 mimetics, anti-diabetic agents, obesity-reducing agents, aldosterone receptor blockers, endothelin receptor blockers, aldosterone synthase inhibitors (ASI), a CETP inhibitor, anti-coagulants, relaxin, BNP (nesiritide) and/or a NEP inhibitor. In some embodiments, the additional therapeutic agent is an ACE inhibitor, relaxin, a natriuretic peptide, ghrelin, and other bioactive peptides (such as disclosed in, e.g., WO2018075822; Erdmann, 2008; and Chakrabarti, 2016, each of which are hereby incorporated by reference herein by in its entirety). In some embodiments, the additional therapeutic agent comprises valsartan, candesartan, or losartan. In some embodiments, the oral drug dosage form further comprises one or more of resveratrol, curcumin, and carnitine.

### C. Methods of Making

In some aspects, provided herein are methods of making the oral formulations and oral dosage forms described herein.

In some embodiments, the method of making the oral formulation comprises admixing the carbohydrate matrix comprising the polysaccharide, the cross-linking agent, the biologically active peptide, the poloxamer, DSPC, and DPPC, thereby obtaining the oral formulation. In some embodiments, the method further comprises admixing the PEG and/or cholesterol with the carbohydrate matrix comprising the polysaccharide, the cross-linking agent, the biologically active peptide, the poloxamer, DSPC, and DPPC. In some embodiments, the method of making the oral formulation comprises admixing pre-determined weight percentages of the carbohydrate matrix, the lipid-based nanoparticle, and the cross-linking agent, wherein: (i) the weight percentage of the carbohydrate matrix comprising the polysaccharide relative to the polysaccharide, the cross-linking agent, the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about 48% to about 98%; (ii) the weight percentage of the cross-linking agent relative to the polysaccharide, the cross-linking agent, the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about 1% to about 5%; and (iii) the weight percentage of the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), of the lipid-based nanoparticles relative to the polysaccharide, the cross-linking agent, the biologically active peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about 1% to 49%.

In some embodiments, the method of making the oral formulation comprises use of a lipid-based nanoparticle, such as a liposome, comprising a weight percentage of the apelin peptide of between about 23% and 27%, a weight percentage of the poloxamer (e.g., poloxamer 188) of between about 6.3% and 10.3%, a weight percentage of DSPC of between about 23% and about 27%, a weight percentage of DPPC of between about 23% and about 27%, and a weight percentage of the PEG (e.g., PEG 8000) of between about 14.7% and about 18.7%. In some embodiments, the method of making the oral formulation comprises use of a lipid-based nanoparticle, such as a liposome, comprising a weight percentage of the apelin peptide of between about 43% and 47%, a weight percentage of the poloxamer (e.g., poloxamer 188) of between about 13% and about 17%, a weight percentage of DSPC of between about 8% and about 12%, a weight percentage of DPPC of between about 8% and about 12%, a weight percentage of the PEG (e.g., PEG 8000) of between about 13% and about 17%, and weight percentage of cholesterol of between about 3% and about 7%.

In some embodiments, the oral formulation is prepared using a spraying, such as spray-drying, technique or a microemulsion technique.

In some embodiments, provided herein is a method of making an oral formulation described herein, the method comprising: (a) dissolving an amount of a material comprising a polysaccharide; (b) admixing a biologically active peptide and a poloxamer in the dissolved material comprising the polysaccharide; (c) spray drying the solution resulting from step (b); and (d) suspending the particles produced from step (c) in a solution of DSPC, DPPC, and a cross-linking agent (and optionally a PEG and/or cholesterol), thereby making the oral formulation.

In some embodiments, provided herein is a method of making an oral formulation described herein, the method comprising: (a) obtaining a solution comprising a plurality of lipid-based nanoparticles; and (b) admixing the lipid-based nanoparticle solution with a carbohydrate matrix comprising a polysaccharide, wherein the admixing is performed at a temperature of about 40° C. to about 80° C., thereby making the oral formulation. In some embodiments, the admixing is performed by spraying the lipid-based nanoparticle solution into the carbohydrate matrix. In some embodiments, the lipid-based nanoparticle solution comprises a cross-linking agent. In some embodiments, the method further comprises admixing the carbohydrate matrix embedded with lipid-based nanoparticles and a cross-linking agent.

In some embodiments, provided herein is a method of making an oral formulation described herein, the method comprising: (a) dissolving an amount of a material comprising a polysaccharide; (b) admixing the biologically active peptide and the poloxamer in the dissolved material comprising the polysaccharide; (c) forming an emulsion of the solution resulting from step (b); and (d) admixing the emulsion from step (c) with a solution of DSPC, DPPC, and a cross-linking agent (and optionally a PEG and/or cholesterol), thereby making the oral formulation.

In some embodiments, the oral dosage form is produced by packaging an amount of an oral formulation described herein in a suitable oral dosage form vehicle, such as a vegetable- or gelatin-based capsule. In some embodiments, the amount of the oral formulation packaged in a suitable oral dosage form vehicle is based on the desired amount of the biologically active peptide per oral dosage form.

### D. Methods of Use

In some aspects, provided herein are methods of using the oral formulations and oral dosage forms described herein. In some embodiments, the use is a pharmaceutical use. In some embodiments, the use is a nutraceutical or bioceutical use. In some embodiments, the method comprises administering to an individual an effective amount of an oral dosage form described herein.

In some embodiments, provided herein is a method of treating and/or preventing a disease or condition in an individual, the method comprising administering to an individual an oral dosage form described herein.

In some embodiments, the disease is a cardiovascular-related disease. In some embodiments, the cardiovascular-related diseases is a cardiac disease, vascular disease, or metabolic disease. In some embodiments, the cardiac diseases is chronic heart failure, acute decompensated heart failure, post-myocardial infarction, atrial fibrillation, Brugada syndrome, ventricular tachycardia, atherosclerosis, ischemic cardiovascular disease, cardiomyopathy, cardiac fibrosis, cardiac ischemia/reperfusion injury, arrhythmia, or amyloidosis. In some embodiments, the vascular diseases is hypertension, resistant hypertension, pulmonary hypertension, peripheral arterial disease, erectile dysfunction, restenosis, or preeclampsia. In some embodiments, the metabolic diseases is Type 2 diabetes, Type 1 diabetes, diabetic nephropathy, diabetic retinopathy, chronic kidney disease, acute kidney disease, renal fibrosis, renal ischemia/reperfusion injury, polycystic kidney disease, hemodialysis, or obesity.

In some embodiments, the cardiovascular-related disease is selected from the group consisting of pulmonary hypertension, heart failure, myocardial infarction, diabetic nephropathy, chronic kidney disease, acute kidney disease, erectile dysfunction, diabetes, and metabolic-related disorders.

In some embodiments, the condition is a water retention-associated condition. In some embodiments, the condition is a burn injury.

### EXEMPLARY EMBODIMENTS

Embodiment 1. An oral formulation of a biologically active peptide comprising a plurality of particles, wherein each particle comprises a carbohydrate matrix comprising a polysaccharide, a cross-linking agent, and a plurality of lipid-based nanoparticles embedded in the carbohydrate matrix, and wherein the lipid-based nanoparticle comprises the biologically active peptide, a poloxamer, 1,2-distearoyl-sn-glycero-3-phosphocholine (DSPC), and 2-dipalmitoyl-sn-glycero-3-phosphocholine (DPPC).

Embodiment 2. The oral formulation of embodiment 1, wherein the biologically active peptide comprises a stretch of at least about 15 contiguous amino acids having a net hydrophobic characteristic.

Embodiment 3. The oral formulation of embodiment 1 or 2, wherein the biologically active peptide comprises a stretch of at least about 10 contiguous amino acids having a net positive charge at pH 7.

Embodiment 4. The oral formulation of embodiment 3, wherein the biologically active peptide comprises, from N- to C-terminus, the stretch of amino acids having a net hydrophobic characteristic and the stretch of amino acids having a net positive charge.

Embodiment 5. The oral formulation of any one of embodiments 1-4, wherein the lipid-based nanoparticles are liposomes comprising a lipid bilayer encapsulating a liquid core.

Embodiment 6. The oral formulation of embodiment 5, wherein each liposome comprises a plurality of the biologically active peptide, wherein a first subset of the plurality of the biologically active peptide is configured such that one portion of the biologically active peptide is embedded in the lipid bilayer and another portion of the biologically active peptide is presented on the outer surface of the lipid bilayer or the inner surface of the lipid bilayer facing the liquid core, wherein the portion of the biologically active peptide embedded in the lipid bilayer is the stretch of amino acids having a net hydrophobic characteristic, and wherein the portion of the biologically active peptide presented on the outer surface of the lipid bilayer or the inner surface of the lipid bilayer facing the liquid core is the stretch of amino acids having a net positive charge.

Embodiment 7. The oral formulation of embodiment 5 or 6, wherein the liquid core comprises a second subset of the plurality of the biologically active peptide.

Embodiment 8. The oral formulation of any one of embodiments 1-7, wherein the biologically active peptide is an apelin peptide.

Embodiment 9. The oral formulation of embodiment 8, wherein the apelin peptide is selected from the group consisting of apelin-12, apelin-13, pyroglutamyl apelin-13 ([Pyrl]-apelin-13]), apelin-17, apelin-19, and apelin-36.

Embodiment 10. The oral formulation of any one of embodiments 1-19, wherein the weight percentage of the biologically active peptide in the lipid-based nanoparticles is about 15% to about 60%.

Embodiment 11. The oral formulation of any one of embodiments 1-10, wherein the poloxamer is poloxamer 188, poloxamer 124, poloxamer 181, poloxamer 184, poloxamer 331, and poloxamer 407, or any combination thereof.

Embodiment 12. The oral formulation of any one of embodiments 1-11, wherein the weight percentage of the poloxamer in the lipid-based nanoparticles is about 1% to about 20%.

Embodiment 13. The oral formulation of any one of embodiments 1-12, wherein the weight percentage of DSPC in the lipid-based nanoparticles is about 5% to about 30%.

Embodiment 14. The oral formulation of any one of embodiments 1-13, wherein the weight percentage of DPPC in the lipid-based nanoparticles is about 5% to about 30%.

Embodiment 15. The oral formulation of any one of embodiments 1-14, wherein the lipid-based nanoparticle further comprises a polyethylene glycol (PEG).

Embodiment 16. The oral formulation of embodiment 15, wherein the average molecular weight of the PEG is about 200 Da to about 20000 Da.

Embodiment 17. The oral formulation of embodiment 15 or 16, wherein the average molecular weight of the PEG is about 8000 Da.

Embodiment 18. The oral formulation of any one of embodiments 15-17, wherein the weight percentage of the PEG in the lipid-based nanoparticles is about 10% to about 20%.

Embodiment 19. The oral formulation of any one of embodiments 1-18, wherein the lipid-based nanoparticle further comprises cholesterol.

Embodiment 20. The oral formulation of embodiment 19, wherein the weight percentage of cholesterol in the lipid-based nanoparticles is about 0.1% to about 10%.

Embodiment 21. The oral formulation of any one of embodiments 1-20, wherein the lipid-based nanoparticle further comprises at least one additional therapeutic agent.

Embodiment 22. The oral formulation of any one of embodiments 15-21, wherein the lipid-based nanoparticle comprises a weight percentage of the apelin peptide of about 25%, a weight percentage of poloxamer 188 of about 8.3%, a weight percentage of DSPC of about 25%, a weight percentage of DPPC of about 25%, and a weight percentage of PEG 8000 of about 16.7%.

Embodiment 23. The oral formulation of any one of embodiments 19-21, wherein the lipid-based nanoparticle comprises a weight percentage of the apelin peptide of about 45%, a weight percentage of poloxamer 188 of about 15%, a weight percentage of DSPC of about 10%, a weight percentage of DPPC of about 10%, a weight percentage of PEG 8000 of about 15%, and weight percentage of cholesterol of about 5%.

Embodiment 24. The oral formulation of any one of embodiments 1-23, wherein the weight percentages of the non-solvent components in the carbohydrate matrix comprising the polysaccharide, the cross-linking agent, and the lipid-based nanoparticles is as follows: the carbohydrate matrix comprising the polysaccharide is about 48% to about 98%, the cross-linking agent is about 1% to about 5%, and the lipid-based nanoparticle is about 1% to 49%.

Embodiment 25. The oral formulation of any one of embodiments 1-24, wherein the size range of the plurality of particles is about 1 μm to about 40 μm.

Embodiment 26. The oral formulation of any one of embodiments 1-25, wherein each of the plurality of particles comprises a plurality of pores.

Embodiment 27. The oral formulation of any one of embodiments 1-26, wherein the polysaccharide is a pectin, gara gum, oak milk carbohydrate, or banana carbohydrate.

Embodiment 28. The oral formulation of embodiment 27, wherein the pectin is a citrus peel pectin.

Embodiment 29. The oral formulation of embodiment 27 or 28, wherein the pectin is 150-grade pectin.

Embodiment 30. The oral formulation of any one of embodiments 1-29, wherein the cross-linking agent is selected from a divalent or polyvalent cation.

Embodiment 31. The oral formulation of embodiment 30, wherein the divalent or polyvalent cation is selected from Ca²⁺, Zn²⁺, Pb²⁺, Cu²⁺, Ba²⁺, Sr²⁺, Cd⁺², Co²⁺, Ni²⁺, or a combination thereof.

Embodiment 32. The oral formulation of any one of embodiments 1-31, wherein the biologically active peptide has a bioavailability in an individual of about 2% or greater.

Embodiment 33. The oral formulation of any one of embodiments 1-32, wherein the plurality of particles is not a gel or hydrogel.

Embodiment 34. An oral dosage form comprising the oral formulation of any one of embodiments 1-33.

Embodiment 35. The oral dosage form of embodiment 34, comprising about 0.1 mg to about 0.5 mg of the biologically active peptide.

Embodiment 36. The oral dosage form of embodiment 34 or 35, further comprising an acceptable excipient.

Embodiment 37. The oral dosage form of any one of embodiments 34-36, wherein the oral dosage form is a tablet, capsule, or caplet.

Embodiment 38. A method of treating and/or preventing a disease in an individual, the method comprising administering to an individual the oral dosage form of any one of embodiments 34-37.

Embodiment 39. A method of making the oral formulation of any one of embodiments 1-34, the method comprising admixing the carbohydrate matrix comprising the polysaccharide, the cross-linking agent, the biologically active peptide, the poloxamer, DSPC, and DPPC, thereby obtaining the oral formulation.

Embodiment 40. The method of embodiment 39, wherein the method further comprises admixing the PEG and/or cholesterol with the carbohydrate matrix comprising the polysaccharide, the cross-linking agent, the biologically active peptide, the poloxamer, DSPC, and DPPC.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

### Example 1

This example demonstrates preparation of liposomes comprising an apelin peptide, a poloxamer, a PEG, DSPC, and DPPC.

DSPC and DPPC were reconstituted in ethanol and sonicated until completely dissolved (the minimum amount of ethanol need to dissolve DSPC and DPPC was used). PEG 8000 and Poloxamer 188 were reconstituted in ethanol and sonicated until completely dissolved. The DSPC and DPPC solution was mixed with the PEG 800 and Poloxamer 188 solution in a single vial. Then, the mixed solution was subjected to nitrogen to remove the solvent. The final solid was dried in vacuum for 3 hours. The lipid film was dissolved in citric acid (300 mmol) solution. The film was suspended for 15 minutes and then filtered with a polycarbonate filter (0.2 nm size). The mixture was exchanged with distilled water by dialysis and then lyophilized. Apelin (180 mg) was then dissolved in distilled water and added to the lipid film. Additional water was added while slowly mixing the solution for about 30 minutes to 1 hour. The formed liposomes were then incubated at 37° C. for 90 minutes prior to lyophilization.

### Example 2

This example demonstrates preparation techniques for oral formulations of an apelin peptide comprising pectin, a poloxamer, DSPC, DPPC, and calcium chloride.

The oral formulation was prepared by a spray drying technique. 5 mg of pectin was weighed and dissolved in 100 mL of water by slow addition of pectin in small portions to a stirring solution of water. Stirring was continued overnight to obtain a viscous solution of 5% pectin. 200 mg of the apelin peptide and 2 g of the poloxamer were added to the pectin solution. The solution was diluted by adding 800 mL of water followed by adding 200 mL of ethanol. The solution was stirred to obtain a homogeneous solution. The solution was then spray dried using the following settings: an inlet temperature of 60° C., aspirator set to 90-95, and a condenser temperature set to 4° C. The accumulated particles were transferred in the collection vessel to a desiccator. 5 g of the particles were suspended in a solution containing 500 mg of DSPC, 500 mg of DPPC, and 200 mg of calcium chloride in acetone. The suspension was stirred overnight. Subsequently, the acetone was evaporated under vacuum using a rotavapor. The resulting formulation was used to produce a calculated amount of the dosage for animal administration. Particles were suspended in water just before the oral administration.

The oral formulation was prepared by a microemulsion technique. 5 mg pectin was weighed and dissolved in 100 mL of water by slow addition of pectin in small portions to a stirring solution of water. Stirring was continued overnight to obtain a viscous solution of 5% pectin. 200 mg of the apelin peptide and 2 g of the poloxamer were added to the pectin solution. The solution was diluted by adding 100 mL of water followed by adding 800 mL of dichloromethane (DCM). The solution was stirred to obtain an emulsion. The emulsion was added to a solution containing 500 mg of DSPC, 500 mg of DPPC, and 200 mg of calcium chloride in acetone. The suspension was stirred overnight. The solvent was evaporated under vacuum using a rotavapor. The resulting formulation was used to produce a calculated amount of the dosage for animal administration. Particles were suspended in water just before the oral administration.

What is claimed is:

1. An oral formulation of a biologically active peptide comprising a plurality of particles,
wherein each particle comprises a carbohydrate matrix comprising a polysaccharide, a cross-linking agent, and a plurality of lipid-based nanoparticles embedded in the carbohydrate matrix, and
wherein the lipid-based nanoparticle comprises the biologically active peptide, a poloxamer, 1,2-distearoyl-sn-glycero-3-phosphocholine (DSPC), and 2-dipalmitoyl-sn-glycero-3-phosphocholine (DPPC).

2. The oral formulation of claim 1, wherein the biologically active peptide comprises a stretch of at least about 15 contiguous amino acids having a net hydrophobic characteristic.

3. The oral formulation of claim 1 or 2, wherein the biologically active peptide comprises a stretch of at least about 10 contiguous amino acids having a net positive charge at pH 7.

4. The oral formulation of claim 3, wherein the biologically active peptide comprises, from N- to C-terminus, the stretch of amino acids having a net hydrophobic characteristic and the stretch of amino acids having a net positive charge.

5. The oral formulation of any one of claims 1-4, wherein the lipid-based nanoparticles are liposomes comprising a lipid bilayer encapsulating a liquid core.

6. The oral formulation of claim 5, wherein each liposome comprises a plurality of the biologically active peptide, wherein a first subset of the plurality of the biologically active peptide is configured such that one portion of the biologically active peptide is embedded in the lipid bilayer and another portion of the biologically active peptide is presented on the outer surface of the lipid bilayer or the inner surface of the lipid bilayer facing the liquid core, wherein the portion of the biologically active peptide embedded in the lipid bilayer is the stretch of amino acids having a net hydrophobic characteristic, and wherein the portion of the biologically active peptide presented on the outer surface of the lipid bilayer or the inner surface of the lipid bilayer facing the liquid core is the stretch of amino acids having a net positive charge.

7. The oral formulation of claim 5 or 6, wherein the liquid core comprises a second subset of the plurality of the biologically active peptide.

8. The oral formulation of any one of claims 1-7, wherein the biologically active peptide is an apelin peptide.

9. The oral formulation of claim 8, wherein the apelin peptide is selected from the group consisting of apelin-12, apelin-13, pyroglutamyl apelin-13 ([Pyrl]-apelin-13]), apelin-17, apelin-19, and apelin-36.

10. The oral formulation of any one of claims 1-9, wherein the weight percentage of the biologically active peptide in the lipid-based nanoparticles is about 15% to about 60%.

11. The oral formulation of any one of claims 1-10, wherein the poloxamer is poloxamer 188, poloxamer 124, poloxamer 181, poloxamer 184, poloxamer 331, and poloxamer 407, or any combination thereof.

12. The oral formulation of any one of claims 1-11, wherein the weight percentage of the poloxamer in the lipid-based nanoparticles is about 1% to about 20%.

13. The oral formulation of any one of claims 1-12, wherein the weight percentage of DSPC in the lipid-based nanoparticles is about 5% to about 30%.

14. The oral formulation of any one of claims 1-13, wherein the weight percentage of DPPC in the lipid-based nanoparticles is about 5% to about 30%.

15. The oral formulation of any one of claims 1-14, wherein the lipid-based nanoparticle further comprises a polyethylene glycol (PEG).

16. The oral formulation of claim 15, wherein the average molecular weight of the PEG is about 200 Da to about 20000 Da.

17. The oral formulation of claim 15 or 16, wherein the average molecular weight of the PEG is about 8000 Da.

18. The oral formulation of any one of claims 15-17, wherein the weight percentage of the PEG in the lipid-based nanoparticles is about 10% to about 20%.

19. The oral formulation of any one of claims 1-18, wherein the lipid-based nanoparticle further comprises cholesterol.

20. The oral formulation of claim 19, wherein the weight percentage of cholesterol in the lipid-based nanoparticles is about 0.1% to about 10%.

21. The oral formulation of any one of claims 1-20, wherein the lipid-based nanoparticle further comprises at least one additional therapeutic agent.

22. The oral formulation of any one of claims 15-21, wherein the lipid-based nanoparticle comprises a weight percentage of the apelin peptide of about 25%, a weight percentage of poloxamer 188 of about 8.3%, a weight percentage of DSPC of about 25%, a weight percentage of DPPC of about 25%, and a weight percentage of PEG 8000 of about 16.7%.

23. The oral formulation of any one of claims 19-21, wherein the lipid-based nanoparticle comprises a weight percentage of the apelin peptide of about 45%, a weight percentage of poloxamer 188 of about 15%, a weight percentage of DSPC of about 10%, a weight percentage of DPPC of about 10%, a weight percentage of PEG 8000 of about 15%, and weight percentage of cholesterol of about 5%.

24. The oral formulation of any one of claims 1-23, wherein the weight percentages of the non-solvent components in the carbohydrate matrix comprising the polysaccharide, the cross-linking agent, and the lipid-based nanoparticles is as follows: the carbohydrate matrix comprising the polysaccharide is about 48% to about 98%, the cross-linking agent is about 1% to about 5%, and the lipid-based nanoparticle is about 1% to 49%.

25. The oral formulation of any one of claims 1-24, wherein the size range of the plurality of particles is about 1 μm to about 40 μm.

26. The oral formulation of any one of claims 1-25, wherein each of the plurality of particles comprises a plurality of pores.

27. The oral formulation of any one of claims 1-26, wherein the polysaccharide is a pectin, gara gum, oak milk carbohydrate, or banana carbohydrate.

28. The oral formulation of claim 27, wherein the pectin is a citrus peel pectin.

29. The oral formulation of claim 27 or 28, wherein the pectin is 150-grade pectin.

30. The oral formulation of any one of claims 1-29, wherein the cross-linking agent is selected from a divalent or polyvalent cation.

31. The oral formulation of claim 30, wherein the divalent or polyvalent cation is selected from Ca²⁺, Zn²⁺, Pb²⁺, Cu²⁺, Ba²⁺, Sr²⁺, Cd⁺², Co²⁺, Ni²⁺, or a combination thereof.

32. The oral formulation of any one of claims 1-31, wherein the biologically active peptide has a bioavailability in an individual of about 2% or greater.

33. The oral formulation of any one of claims 1-32, wherein the plurality of particles is not a gel or hydrogel.

34. The oral formulation of any one of claims 1-33, produced using a spray technique and/or microemulsion technique.

35. An oral dosage form comprising the oral formulation of any one of claims 1-34.

36. The oral dosage form of claim 35, comprising about 0.1 mg to about 0.5 mg of the biologically active peptide.

37. The oral dosage form of claim 35 or 36, further comprising an acceptable excipient.

38. The oral dosage form of any one of claims 35-37, wherein the oral dosage form is a tablet, capsule, or caplet.

39. A method of treating and/or preventing a disease in an individual, the method comprising administering to an individual the oral dosage form of any one of claims 35-38.

40. A method of making the oral formulation of any one of claims 1-34, the method comprising admixing the carbohydrate matrix comprising the polysaccharide, the cross-linking agent, the biologically active peptide, the poloxamer, DSPC, and DPPC, thereby obtaining the oral formulation.

41. The method of claim 40, wherein the method further comprises admixing the PEG and/or cholesterol with the carbohydrate matrix comprising the polysaccharide, the cross-linking agent, the biologically active peptide, the poloxamer, DSPC, and DPPC.
